(19) United States
(12) Patent Application Publication
(10) Pub. No.: US 2007/0249912 A1
(43) Pub. Date: Oct. 25, 2007

(54) METHOD FOR ARTERY-VEIN IMAGE SEPARATION IN BLOOD POOL CONTRAST AGENTS

(75) Inventor: Huseyin Tek, Princeton, NJ (US)

Correspondence Address: SIEMENS CORPORATION;INTELLECTUAL PROPERTY DEPARTMENT, 170 WOOD AVENUE SOUTH, ISELIN, NJ, 08830, US

(73) Assignee: Siemens Corporate Research, Inc., Princeton, NJ (US)

(21) Appl. No.: 11/676,337

(22) Filed: Feb. 19, 2007

Related U.S. Application Data
(60) Provisional application No. 60/793,860, filed on Apr. 21, 2006.

(51) Int. Cl. A61B 5/00

(52) U.S. Cl. 600/300

(57) ABSTRACT

A method for segmenting and separating arteries and veins in blood pool contrast agents (MRA). Specifically, arteries and veins are accurately segmented by an algorithm that combines local vessel models, discrete centerline models and ordered statistical front propagation to produce accurate segmentation results with the minimum amount of non-vascular inclusion. Separation of arteries and veins is obtained by incorporating centerline models to the distance based watershed transforms.

### CROSS REFERENCE TO RELATED APPLICATION

This application claims priority from U.S. Provisional application Ser. No. 60/793,860 filed on Apr. 21, 2006 which is incorporated herein by reference.

### INCORPORATION BY REFERENCE

This patent application incorporates by reference the following co-pending patent applications:

Ser. No. 10/951,194 entitled “Local Watershed Operators For Image Segmentation”, filed Sep. 27, 2004, inventor Huseyin Tek, assigned to the same assignee as the present invention (Pub. No. US 2005/0201618 A1);

Ser. No. 11/231,424 entitled “Region Competition Via Local Watershed Operation”, filed Sep. 21, 2005, inventor Huseyin Tek, et al., assigned to the same assignee as the present invention (Pub. No. US 2006/0098870 A1); and

Ser. No. 11/399,164 entitled “Method and Apparatus For Detecting Vessel Boundaries”, filed Apr. 6, 2006, inventor Huseyin Tek, assigned to the same assignee as the present invention (Pub. No. US 2006/0262988 A1).

### TECHNICAL FIELD

This invention relates generally to generally to medical diagnostics, and more particularly to the determination of blood vessel boundaries in a medical image (i.e., vessel segmentation). The invention also relates to segmenting blood vessels from vein vessels.

### BACKGROUND

As is known in the art, to diagnose a problem of a patient, medical professionals often have to examine the patient's vessels (e.g., blood vessels). To illuminate a vessel so that the medical professional can examine the vessel, a patient consumes (e.g., drinks) a contrast-enhancing agent. The contrast-enhancing agent brightens one or more vessels relative to the surrounding area.

The main goal of the majority of contrast-enhanced (CE) magnetic resonance angiography (MRA) and computed tomography angiography (CTA) is diagnosis and qualitative or quantitative assessment of pathology in the circulatory system. Once the location of the pathology is determined, quantitative measurements can be made on the original 2 dimensional slice data or, more commonly, on 2 dimensional multi planar reformat (MPR) images produced at user-selected positions and orientations. In the quantification of stenosis, it is often desirable to produce a cross-sectional area/radius profile of a vessel so that one can compare pathological regions to healthy regions of the same vessel.

Accurate and robust detection of vessel boundaries (i.e., vessel segmentation) is traditionally a challenging task. In particular, a vessel boundary detection algorithm has to be accurate and robust so that the algorithm can be used to accurately detect vessel boundaries on many types of medical images. If the vessel boundary detection algorithm is inaccurate (even in a small number of cases), a medical professional (e.g., a radiologist) relying on the computer's output may, in turn, incorrectly diagnose the patient.

There are many reasons why accurate and robust detection of vessel boundaries is a challenging task. First, the presence of significant noise levels in computed tomography (CT) and magnetic resonance (MR) images often forms strong edges (i.e., changes in intensity between data points) inside vessels. Second, the size of a vessel can vary from one vessel location to another, resulting in additional edges. Third, the intensity profile of a vessel boundary can be diffused at one side while shallow on the other sides (e.g., due to the presence of other vessels or high contrast structures). Fourth, the presence of vascular pathologies, e.g., calcified plaques, often makes the shape of a vessel cross-sectional boundary locally deviate from a circular shape. These all result in additional edges that can affect an accurate determination of a vessel boundary.

There have been a variety of techniques that have been used to address the above-mentioned challenges. For example, medical professional have estimated the boundary of a vessel using computer-aided drawing programs. This is an inaccurate process because the estimation of the boundary can vary widely from the actual boundary.

Another example is a “snake” model for segmenting vessel boundaries in the planes orthogonal to the vessel centerline. The “snake” model traditionally “inserts” a tube having a smaller diameter than the vessel into a representation of the vessel and then uses parameters to cause the tube to expand until reaching the vessel's walls. The selection of the parameters, however, is often initially estimated. An inaccurate selection of one or more parameters may result in the tube expanding beyond the actual vessel boundary. Thus, the snake model does not always provide accurate results.

Another attempt to address the above-mentioned challenges is a ray propagation method. This method is based on the intensity gradients for the segmentation of vessels and detection of their centerline. However, the use of gradient strength by itself is often not enough for robust segmentation.

Another approach to solve the above-mentioned problem is based on explicit front propagation via normal vectors, which then combines smoothness constraints with mean-shift filtering. Specifically, the curve evolution equation

$$\frac{d ( C , s )}{d t} = S ( x , y ) \{ {N}^{->} \}$$

was determined for the vessel boundaries where C(s,t) is a contour, S(x,y) is the speed of evolving contour and {\vec{N}} is the vector normal to C(s,t). In this approach, the contour C(s,t) is sampled and the evolution of each sample is followed in time by rewriting the curve evolution equation in vector form. The speed of rays, S(x,y) depends on the image information and shape priors. S(x,y)=Sₒ(x,y)+βS₁(x,y) was proposed where Sₒ(x,y) measures image discontinuities, S₁(x,y) represents shape priors, and β balances these two terms. Image discontinuities are detected via mean-shift analysis along the rays. Mean-shift analysis, which operates in the joint spatial-range domain where the space of the 2 dimensional lattice represents the spatial domain and the space of intensity values constitutes the range domain, is often used for robustly detecting object boundaries in images. This approach is often effective when vessel boundaries are well isolated. It is often difficult, however, to estimate parameters such as spatial, range kernel filter sizes, and/or the amount of smoothness constraints for the robust segmentation of vessels. In particular, the use of a single spatial scale and curvature based smoothness constraints are typically not enough for accurate results when vessels are not isolated very well.

Thus, separation of arteries and veins in blood pool contrast agents (MRA) is a difficult task. Specifically, special types of vessel segmentation algorithms are often required for this problem because arteries and veins often touch each other due to the partial voluming effects and significant amount of bright tissue/structures is present in these blood pool contrast enhanced (CE) MRA data. Previously, several algorithms have been proposed for the artery-vein separation problem. See also: T. Lei, J. K. Udupa, P. K., S. P K, and D. Odhner. Artery-vein separation via MRA—an image processing approach. IEEE Trans. Medical Imaging, 20(8):689-703, 2001l; R. M. Stefancik and M. Sonka. Highly automated segmentation of arterial and venous trees from three-dimensional magnetic resonance angiography (MRA). Int. J. of Cardiac Imaging, 17(1):37-47, 2001; and C. M. van Bemmel, L. J. Spreeuwers, M. A. Viergever, and W. J. Niessen. Level-set-based artery-vein separation in blood pool agent CE-MR angiograms. IEEE Trans. on Medical Imaging, 22(10): 1224-11234, 2003.

### SUMMARY

In accordance with the present invention, a method is provided for artery segmentation from background in a patient. The method includes: performing modeling on a local vessel of the patient; applying discrete centerline models to the modeled local vessel; generating an ordered statistical front propagation on the discrete centerline models; and generating arteries and veins from the generating ordered statistical front propagation generated on the discrete centerline models.

In one embodiment, the method includes separating, in the generated arteries and veins, the arteries from the veins using centerline models to the distance based watershed transforms.

In accordance with another feature of the invention, a method is provided for artery segmentation from background in a patient. The method includes: local vessel modeling; discrete centerline models; and ordered statistical front propagation. Next, the method separates arteries and veins using centerline models to the distance based watershed transforms.

In one embodiment, the local vessel modeling; discrete centerline models; and ordered statistical front propagation includes partial vessel segmentation and ordered front propagation.

In one embodiment, the partial vessel segmentation includes: segmenting the vessels using local vessel modeling; and developing statistical front propagation and modeling from the local vessel modeling.

In one embodiment, the segmenting of the vessels using local vessel modeling comprises: placing seed points on an image of a portion of the patient.

In one embodiment, the developing statistical front propagation and modeling from the local vessel modeling for the each seed point comprises: locally estimating vessel and surrounding background statistics by computing vessel orthogonal planes and corresponding cross-sectional boundaries; and segmenting vessels in a limited area partially based on a front propagation algorithm using the locally estimated statistics.

In one embodiment, the method includes using ordered front propagation comprising applying discrete centerline modeling using the developed statistical front propagation and modeling comprising re-estimating vessel statistics using discrete fronts and surface filling.

In one embodiment, the ordered front propagation includes applying discrete centerline modeling using the developed statistical front propagation and modeling comprising re-estimating vessel statistics using discrete fronts and surface filling followed by determining a measure of accuracy of each front using a discrete centerline model obtained by minimal path detection operating a distance map and re-starting partial segmentation from the front having the highest confidence measure representing the correct vessel.

In one embodiment, the method iteratively performs the partial segmentation and the ordered front propagation to segment arterial and venous vessels independently; each one of the arterial and venous vessels having a separate arterial and venous vessels map.

In one embodiment, the method combines the independently segmented arterial and venous vessels maps into a single map. The arterial and venous vessels in the singe map are then separated by a distance-based watershed transform using discrete centerline models between seeds used as the markers for the watershed transforms.

In accordance with the present invention, a method is provided for artery segmentation from background in a patient, comprising: segmenting the vessels using local vessel modeling; developing statistical front propagation and modeling from the local vessel modeling; and using discrete centerline modeling.

In accordance with another feature of the invention, a method is provided for artery segmentation from background in a patient. The method includes: local vessel modeling; discrete centerline models; and ordered statistical front propagation. Separation of arteries and veins is obtained using centerline models to the distance based watershed transforms.

In one embodiment, the segmenting of the vessels using local vessel modeling comprises: placing seed points on an image of a portion of the patient.

In one embodiment, developing statistical front propagation and modeling from the local vessel modeling for the each seed point comprises: locally estimating vessel and surrounding background statistics by computing vessel orthogonal planes and corresponding cross-sectional boundaries; segmenting vessels in a limited area partially based on a front propagation algorithm using the estimated statistics.

In one embodiment, the discrete centerline modeling comprises re-estimating vessel statistics using discrete fronts and surface filling.

In one embodiment, the method determines a measure of accuracy of each front using a discrete centerline model obtained by minimal path detection operating a distance map and re-starts partial segmentation from the front having the highest confidence measure representing the correct vessel. The process iteratively performing the partial segmentation process to segment arterial and venous vessels independently; each one of the arterial and venous vessels having a separate arterial and venous vessels map.

In one embodiment, the method combines the independently segmented arterial and venous vessels maps into a single map. The arterial and venous vessels in the single map are then separated by a distance-based watershed transform using discrete centerline models between seeds used as the markers for the watershed transforms.

By re-starting partial segmentation from the front that has the highest confidence measure of representing the correct vessel, there is minimal segmentation of non-vascular bright structures since they often cannot form a tubular centerline models. The propagation of fronts are stopped when the pre-determined topology between seed points is established or when there are no valid fronts. This iterative partial segmentation process is applied to the segmentation of arterial and venous, independently.

Further, instead of using seeds in the watershed transform, discrete centerline models between the seeds are used as the markers for the watershed transforms for the increased accuracy.

The method produces segmentation results with high accuracy. Specifically, the local estimation of vessel intensity statistics via vessel models allows good vessel boundary delineation. In addition, minimal amount of non-vascular structures are present in the results due to the use of local centerline models during the segmentation process. Furthermore, partial segmentation and modeling allows segmentation of vessels with significant contrast changes along them. The separation of segmented arteries and veins via distance-based watershed transforms via centerline produce very accurate results even when arteries and veins touch each other in many places.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a flow chart of a method for artery segmentation from background in a patient according to the invention;

FIGS. 2(a)-2(e) illustrates the cross-sectional boundary extraction algorithm step by step on a CE-MRA data used in the method of FIG. 1; FIG. 2(a) showing multi-scale edges are detected along 1D rays, FIG. 2(b) showing correct edges after deletion of incorrect edges, FIG. 2(c) showing prominent edge selection, FIG. 2(d) showing curve segments obtained from local edge grouping algorithm, and FIG. 2(e) showing vessel boundary obtained from an elliptical fit;

FIGS. 3(a)-3(c) illustrates the mean-shift filtering of a typical edge; FIG. 3(a) showing the original intensity profile; FIG. 3(b) showing displacement vectors where divergence of displacement vectors corresponds to the location of the edge; and FIG. 3(c) showing smoothed intensity and original intensity together and the two local mode of intensity (convergence points) around the edge;

FIG. 4 illustrates an example where the vicinity of a seed point is segmented by the method according to the invention; more particularly, FIG. 4 illustrates the partial vessel segmentation results (labeled GREEN) from a seed point and the corresponding discrete fronts (labeled RED);

FIG. 5 illustrates the discrete line models (labeled BLUE) between seed points and discrete fronts (labeled RED), more particularly; FIG. 5 illustrates the discrete centerline (labeled BLUE) between the source and each discrete front. Since these connected centerlines are in 3D, they may appear broken in 2D visualization. In addition, centerlines are dilated for better visualization;

FIGS. 6(a)-6(f) illustrate the results of artery vein separation algorithm according to the invention on two different MRA data sets on two different MRA data; FIGS. 6(a) and 7(d) showing the arteries (labeled RED) and veins (labeled BLUE) depicted in volume rendering (VR) visualization; FIGS. 6(b) and 6(e) showing a venous map; and,

FIGS. 6(c) and 6(f) showing an arterial map;

FIG. 7 is a schematic diagram of a computer configured to perform the method according to the invention; and

FIGS. 8A and 8B show a plane passing through a vessel with such plane passing therethrough orthogonal to the centerline of the vessel.

Like reference symbols in the various drawings indicate like elements.

### DETAILED DESCRIPTION

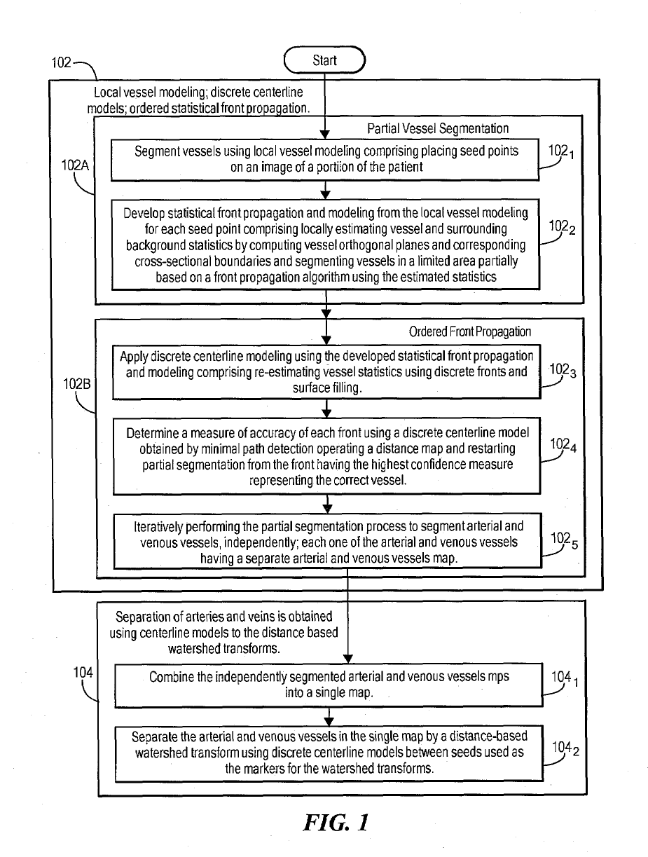

Referring now to FIG. 1 a flow chart is shown of a method for artery segmentation from background in a patient. The method includes: local vessel modeling; discrete centerline models; and ordered statistical front propagation, Step 102. Next, the method separates arteries and veins using centerline models to the distance based watershed transforms, Step 104.

More particularly, the local vessel modeling; discrete centerline models; and ordered statistical front propagation, Step 102 includes partial vessel segmentation, Step 102A and ordered front propagation, Step 102B.

Partial vessel segmentation, Step 102A includes: segmenting the vessels using local vessel modeling, Step 102₁; and developing statistical front propagation and modeling from the local vessel modeling, Step 102₂. The segmenting of the vessels using local vessel modeling (Step 102₁) comprises: placing seed points on an image of a portion of the patient. The developing statistical front propagation and modeling from the local vessel modeling for the each seed point (Step 102₂) comprises: locally estimating vessel and surrounding background statistics by computing vessel orthogonal planes and corresponding cross-sectional boundaries; and segmenting vessels in a limited area partially based on a front propagation algorithm using the estimated statistics.

Next, the method uses ordered front propagation; Step 102B using discrete centerline modeling comprises re-estimating vessel statistics using discrete fronts and surface filling. More particularly, ordered front propagation, Step 102B, includes applying discrete centerline modeling using the developed statistical front propagation and modeling comprising re-estimating vessel statistics using discrete fronts and surface filling, Step 102₃. Next, the method determines a measure of accuracy of each front using a discrete centerline model obtained by minimal path detection operating a distance map and re-starts partial segmentation from the front having the highest confidence measure representing the correct vessel, Step 102₄. The process iteratively performing the partial segmentation process (i.e., Steps 102₁-102₂) and Steps 102₃-102₄ of the ordered front propagation process to segment arterial and venous vessels, independently; each one of the arterial and venous vessels having a separate arterial and venous vessels map, Step 102₅.

The method, in Step 104, combines the independently segmented arterial and venous vessels maps into a single map, Step 104₁. The arterial and venous vessels in the singe map are then separated by a distance-based watershed transform using discrete centerline models between seeds used as the markers for the watershed transforms. Step 104₂.

### Partial Vessel Segmentation, Step 102A

Here, the radiologist or user places seed points on an image of a portion of the patient; For each seed point, the process locally estimates vessel and surrounding background statistics by computing vessel orthogonal planes and corresponding cross-sectional boundaries, Step 102₁.

Local background/foreground separation can be accurately accomplished if the parameters of the normal distribution are estimated correctly. The method uses geometric tubular models to estimate these parameters. (Reference is briefly made to FIGS. 8A and 8B which show one orthogonal plane 800 and corresponding cross-sectional boundaries 802 of a vessel 804. Note that the plane 800 is orthogonal to the centerline 806 of the vessel 804.) Specifically, for a given point inside a vessel, first, the direction of vessel is determined from the eigenvalue analysis of Hessian matrix, and second the corresponding vessel cross-sectional boundary is computed. The distribution parameters describing vessel and its immediate surroundings can be easily obtained from this 2D segmentation.

Most popular approach for computing vessel specific coordinate system is based on the eigenvalue analysis of Hessian matrix, e.g., A. F. Frangi, W. J. Niessen, K. L. Vincken, and M. A. Viergever. Multiscale vessel enhancement filtering. In MICCAI, pages 82-89, 1998; O. Wink, W. J. Niessen, and M. A. Viergever. Multiscale vessel tracking. IEEE Trans. on Medical Imaging, 23(1):130-133, 2004; M. Descoteaux, L. Collins, and K. Siddiqi. Geometric flows for segmenting vasculature in mri: Theory and validation. In Medical Image Conference and Computer Assisted Interventions (MICCAI), 2004; and S. Aylward and E. B. E. Initialization, noise, singularities, and scale in height-ridge traversal for tubular object centerline extraction. IEEE Trans. on Medical Imaging, 21(2):61-75, 2002. Typically, a 3D image is filtered with normalized second order Gaussian derivatives to compute the Hessian matrix at each location.

The eigenvalues, λ₁, λ₂, λ₃, corresponds to the derivatives along the eigenvectors, e₁, e₂, e₃ where the eigenvector e₁ corresponding to the smallest eigenvalue is in the direction of tube and the other eigendirections e₁, e₂ defines the cross-sectional plane, thus forming an orthonormal coordinate system. In this paper, we use the vesselness measure proposed in [9] to find the approximate direction of the vessel of interests. Specifically,

$$V ( σ ) = \{ \begin{matrix}0 , & if {λ}_{2} > 0 or {λ}_{3} > 0 \\ ( 1 - {e}^{( - \frac{{R}_{Δ}^{2}}{2 {σ}^{2}} )} ) {e}^{- \frac{{R}_{B}^{2}}{2 {β}^{2}}} ( 1 - {e}^{- \frac{{S}^{2}}{2 {γ}^{2}}} ) & otherwise\end{matrix} \\ where \\ {R}_{A} = \frac{{λ}_{2}}{{λ}_{3}} , {R}_{B} = \frac{{λ}_{1}}{\sqrt{{λ}_{2} {λ}_{3}}} , \\ S = \sqrt{{λ}_{1}^{2} + {λ}_{2}^{2} + {λ}_{3}^{2}} \qquad ( 1 )$$

and α, β and γ are the parameters. Since vessels in 3D images can be in different sizes, images are convolved with multi-scale filters and maximum response is taken as a vesselness measure at point.

The intensity distribution of a partial vessel segment is determined from the 2D cross-sectional boundaries. There have been several techniques for computing vessel cross-sectional boundaries (see O. Wink, W. Niessen, and M. A. Viergever. Fast delination and visualization of vessels in 3-D angiographic images. IEEE Trans. on Medical Imaging, 19:337-345, 2000; M. Hernandez-Hoyos, A. Anwander, M. Orkisz, J. P. Roux, and I. E. M. P. Doueck. A deformable vessel model with single point initialization for segmentation, quantification and visualization of blood vessels in 3D MRA. In MICCAI'00, 2000. and H. Tek, A. Ayvaci, and D. Comaniciu. Multi-scale vessel boundary detection. In Workshop of CVBIA, pages 388-398, 2005. Here, the method uses the technique developed by H. Tek, A. Ayvaci, and D. Comaniciu. Multi-scale vessel boundary detection. In Workshop of CVBIA, pages 388-398, 2005) since it separates the intensities of a vessel and its surrounding areas by a multi-scale mean shift filtering, see for example Ser. No. 11/399,164 entitled “Method and Apparatus For Detecting Vessel Boundaries”, filed Apr. 6, 2006, inventor Huseyin Tek, assigned to the same assignee as the present invention (Pub. No. US 2006/0262988 A1).

Specifically, first, edges along a ray are computed in several scales by using mean shift analysis D. Comaniciu and P. Meer. Mean shift: A robust approach toward feature space analysis. IEEE Trans. PAMI, 24(5):603-619, 2002.

Second, incorrect edges obtained from multiple scales are eliminated by the mean-shift based clustering algorithm.

Third, prominent edges are obtained by selecting edges based on their strengths, and the assumption that vessels are not nested structures.

Fourth, smooth and long curve segments are constructed from prominent edges by a local grouping algorithm.

Finally, the curve segments that best describe the vessel boundary are determined from the elliptic shape priors.

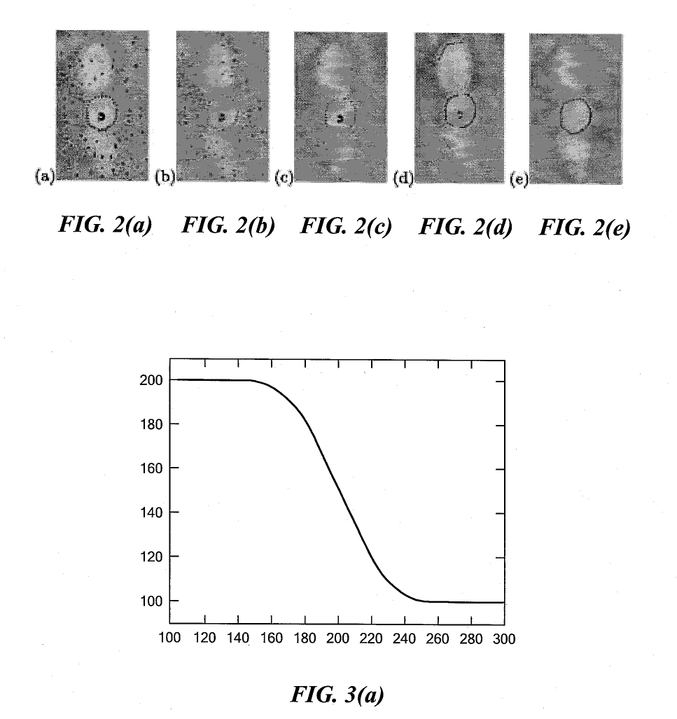

The method is capable of segmenting vessel boundaries in great detail even in the extreme conditions. FIGS. 2(a)-2(e) illustrate the steps of the algorithm on MRA data. In addition to the accurate cross-sectional boundaries, this technique classifies intensities inside the vessel and its background via multi-scale mean shift filtering. More particularly, FIGS. 2(a)-2(d) illustrates the cross-sectional boundary extraction algorithm step by step on a CE-MRA data. FIG. 2(a) shows multi-scale edges are detected along 1D rays, FIG. 2(b) shows correct edges after deletion of incorrect edges. FIG. 2(c) shows prominent edge selection. FIG. 2(d) shows curve segments obtained from local edge grouping algorithm. FIG. 2(e) shows vessel boundary obtained from an elliptical fit.

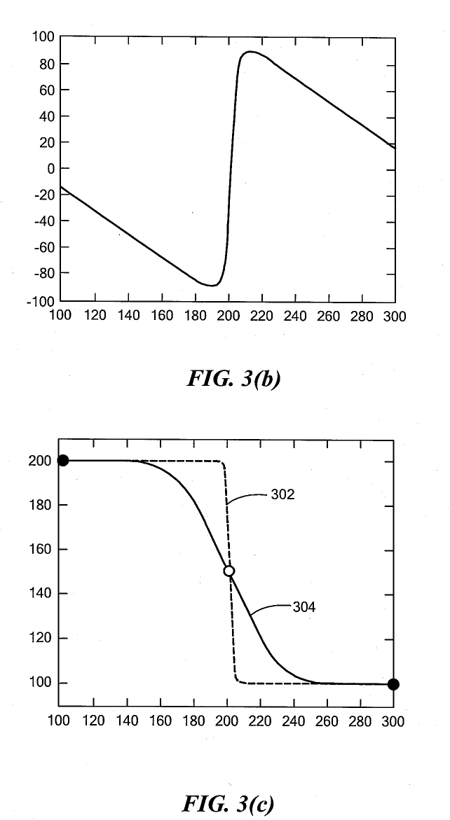

The normal distribution parameters of vessel N(V_{μ},V_{σ}) and its immediate surroundings (or background) N(B_{μ},B_{σ}) are obtained from the local mode of mean-shift filtering. FIGS. 3(a)-3(c) illustrates how mean-shift filtering separates vessel and background. More particularly, FIGS. 3(a)-3(c) illustrates the mean-shift filtering of a typical edge. FIG. 3(a) shows the original intensity profile; FIG. 3(b) shows displacement vectors where divergence of displacement vectors corresponds to the location of the edge. FIG. 3(c) shows smoothed intensity 302 and original intensity together and the two local mode of intensity (convergence points) 304 around the edge. The x-axis of FIGS. 3(a)-3(c) represents distance along an image (i.e., scale) and the y-axis of FIGS. 3(a) and 3(c) represents intensities and the y-axis of FIG. 3(c) represents displacement values as described in more detail in the above referenced Ser. No. 11/399,164 entitled “Method and Apparatus For Detecting Vessel Boundaries”, filed Apr. 6, 2006, inventor Huseyin Tek, assigned to the same assignee as the present invention (Pub. No. US 2006/0262988 A1) incorporated herein by reference.

Next, in Step 102₂, for the each seed point, the method locally estimates vessel and surrounding background statistics by computing vessel orthogonal planes, as shown and described above, for one orthogonal plane 800 in FIGS. 8A and 8B and corresponding cross-sectional boundaries 802 of the vessel 804. As noted above, the plane 800 is orthogonal to the centerline 806 of the vessel 804.

More particularly, once the parameters of normal distribution are determined, accurate partial vessel segmentation can be easily accomplished by region growing or region competition via watershed transforms, see H. Tek, F. Akova, and A. Ayvaci. Region competition via local watershed operators. In CVPR, pages 361-368, 2005, see for example Ser. No. 10/951,194 entitled “Local Watershed Operators For Image Segmentation”, filed Sep. 27, 2004, inventor Huseyin Tek, assigned to the same assignee as the present invention (Pub. No. US 2005/0201618 A1); and Ser. No. 11/231,424 entitled “Region Competition Via Local Watershed Operation”, filed Sep. 21, 2005, inventor Huseyin Tek, et al., assigned to the same assignee as the present invention (Pub. No. US 2006/0098870 A1).

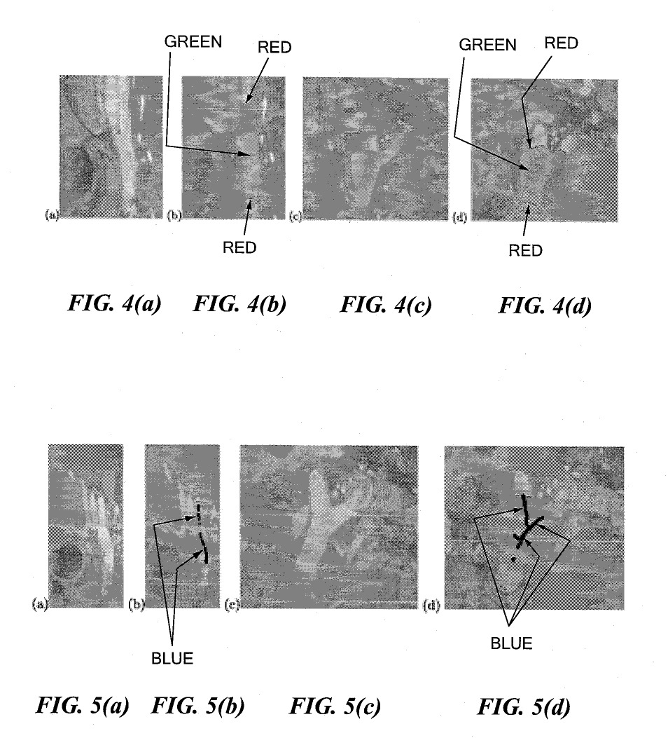

FIG. 4 illustrates an example where the vicinity of a seed point is segmented by this algorithm. More particularly, FIG. 4 illustrates the partial vessel segmentation results (labeled GREEN) from a seed point and the corresponding discrete fronts (labeled RED). (It should be understood that the items labeled RED and GREEN would typically be displayed in colors such as for example red and green, respectively).

Let us now illustrate how these partial segmentation are grouped by the process to obtain segmentation of the whole branch.

### Ordered Front Propagation, Step 102B

Next, in Step 1023, vessel statistics are re-estimated using discrete fronts and surface filling. More particularly, the accuracy of vessel segmentation results by continuing the above process heavily depends on where the segmentation re-starts from. Note that the voxels in binary segmentation obtained from the above algorithm are further classified as converged and alive points. Converged points correspond to the voxels which cannot extend the segmentation because there is no vessel point in their neighborhood. Alive points correspond to voxels that are stopped by the distance constraint and their neighborhood contains voxels that can be classified as vessel. If the alive points were not stopped by the distance constraint, they could have segmented vessels and other bright structures. Thus, the alive points must be grouped and classified whether they represent vessels or non-vascular structures. Specifically, voxels from the alive point list are grouped to each other via discrete connectivities, which are called discrete fronts. FIG. 4 illustrates the discrete fronts labeled RED.

The process used to obtain discrete fronts from the alive point list is as follows: First, all alive points are marked as one in an empty map whose voxel values are set to zero. Second, a point from alive list is selected and the voxels that are connected to it are determined by a simply surface filling process. During the surface filling process, the connected voxels are set to empty and they are removed from the alive list. This process determines a single discrete front. The other fronts are determined by repeating this process until there is no more point left in the alive list. FIG. 4 illustrates the discrete fronts of a partial segmentation.

Next, in Step 102₄, the process re-estimates vessel statistics using discrete fronts and surface filling. More particularly, at this stage, the process has obtained K discrete fronts from which new partial segmentation needs to start. Since not every discrete front corresponds to the correct vessel, the process first assigns a confidence measure to each front. This confidence measure can be computed from the vesselness measure (see A. F. Frangi, W. J. Niessen, K. L. Vincken, and M. A. Viergever. Multiscale vessel enhancement filtering. In MICCAI, pages 82-89, 1998) the surface area and smoothness of the fronts and the characteristics of the centerlines between the discrete front and the source of partial segmentation, namely the seed point. Here, the process first computes the vesselness measure for a point representing the discrete front. If the vesselness measure is relatively high, a confidence measure based on the centerline models is computed. Observe that voxels representing the fronts must be determined before these algorithms are applied. Here, the voxel representing a front best must be located in the center of the front. Algorithmically, the process first computes the distance map of the segmented area starting from the convergence points and then selects the voxels with highest distance value for the each front. After obtaining a representative point for each front, the process computes the discrete centerlines between them and the source (seed), via a minimal path detection algorithm (based on Dijkstra's method). The cost function (or speed function) this algorithm is computed from the distance transform of segmented vessel and the distance between voxels. Specifically, the cost function for a voxel Vⱼ visited by voxel Vᵢ is given by Cost(Vⱼ)=(1/DT(Vⱼ))*dist(Vⱼ,Vᵢ) where DT is the distance transform of the segmented vessels and dist(Vⱼ,Vᵢ) measures the unsigned distance from Vⱼ to Vᵢ. The DT value forces the front to propagate near the vessel centers. A similar algorithm is presented by Deschamps and Cohen (see T. Deschamps and L. Cohen. Fast extraction of minimal paths in 3d images and applications to virtual endoscopy. Medical Image Analysis, 5(4):281-299, 2001) for finding paths in tubular structures such as the colon. This minimum cost path detection algorithm results in a discrete path consisting of ordered discrete voxel locations. FIG. 5 illustrates the discrete line models (labeled BLUE) between seed points and discrete fronts (labeled RED). More particularly, FIG. 5 illustrates the discrete centerline (labeled BLUE) between the source and each discrete front. Since these connected centerlines are in 3D, they may appear broken in 2D visualization. In addition, centerlines are dilated for better visualization. (It should be understood that the items labeled RED and BLUE would typically be displayed in colors such as for example red and blue, respectively).

The process next, in Step 102₅, re-starts partial segmentation from the front having the highest confidence measure representing the correct vessel. More particularly, the discrete centerlines between the source and each discrete front have been obtained. Confidence measure for the each front is then determined from the radius function along their centerlines. Ideally, fronts which represent the continuing vessels with the highest confidence measure should have almost constant radius profile assuming that the partial segmentation is applied to the relatively small area i.e., vessel size does not change drastically. On the other hand, if a front is inside another type of vessel or in a non-vascular structure, its radius profile should have abrupt changes. In fact, partial segmentation often leaks into the bright tissues and other vessels from gaps on the boundary, where radius values are very small. Based on these observations, the process assigns each front a confidence measure based on the smoothness of the radius values along its centerline model. The segmentation process re-starts from the front that has the most confidence measure. The center of the front is marked as virtual seed (or source) and the parameters of the normal distribution of the vessel is recomputed by the method described above. This ordered-based partial segmentation continues until all the connections between the user placed seed points are established or all the discrete fronts are propagated.

### Separation of Arteries and Veins, Step 104

The process iteratively performs the partial segmentation process to segment arterial and venous vessels, independently; each one of the arterial and venous vessels having a separate arterial and venous vessels map. More particularly, the method combines the independently segmented arterial and venous vessels maps into a single map, Step 104₁. The arterial and venous vessels in the singe map are then separated in Step 104₂ by a distance-based watershed transform using discrete centerline models between seeds used as the markers for the watershed transforms.

More particularly, the segmentation process described above is applied for the segmentation of arteries and veins, separately (i.e., independently). The resulting discrete artery (or venous) map often includes areas corresponding to veins (or arteries), thus simple combination of these maps does not suffice for an accurate separation. In this paper, we propose to use the distance based watershed transforms (see L. Vincent and P. Souille. Watersheds in digital spaces: an efficient algorithm based on immersion simulations. PAMI, 13(6):583-598, 1991 and H. Tek and H. C. Aras. Local watershed operators for image segmentation. In Medical Image Computing and Computer-Assisted Intervention MICCAI, pages 127-134, 2004 for the separation of these two maps.) See Ser. No. 10/951,194 entitled “Local Watershed Operators For Image Segmentation”, filed Sep. 27, 2004, inventor Huseyin Tek, assigned to the same assignee as the present invention (Pub. No. US 2005/0201618 A1); and Ser. No. 11/231,424 entitled “Region Competition Via Local Watershed Operation”, filed Sep. 21, 2005, inventor Huseyin Tek, et al., assigned to the same assignee as the present invention (Pub. No. US 2006/0098870 A1).

In image segmentation, the gradient map of images is used as a height map in watershed based segmentation algorithms. Here, the process uses a distance-transform of the combined artery-vein segmentation maps. Specifically, the inverse distance map is quite suitable for the separation of two masks since artery and vein maps often overlap in small areas. In addition, here, the method uses the discrete centerline models between the user placed seeds as the markers for the watershed transforms. These discrete centerline models are computed by a minimal path detection algorithm operating the distance of the segmentation results. Since the user placed seed points are very strong clues for the correct labeling, the separation algorithm should use such information as much as possible. Thus, the distance map is further modified by the addition of a potential function created in the vicinity of the user placed seed points. This potential function for each seed forces the discrete centerline pass away from the other types of vessels.

Furthermore, the validity of the centerline models is verified by reconstructing the vessels from them. If the reconstruction from the arteries and veins overlap significantly, the corresponding centerline models are not used in separation. It should also be noted that it is not always possible to separate arteries and veins in certain areas. This is true especially, when the arteries and veins touch each other over a large region. Such region corresponds to a single basin in watershed map. Thus, here a basin-partitioning algorithm based on distance transforms is used. Specifically, if the user placed seed points or centerline models pass through a same basin, this basin is partitioned based the distance transform from these inputs. This basin partitioning property allows the user to be able to correct any kind of errors by placing additional seeds.

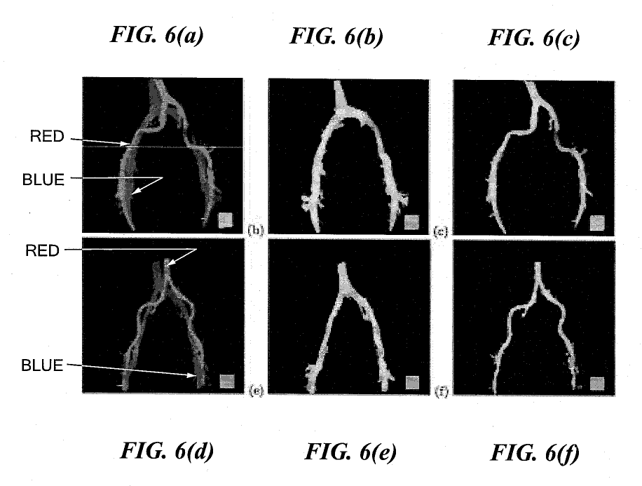

FIGS. 6(a)-6(f) illustrates the results of artery vein separation algorithm according to the invention on two different MRA data sets. These FIGS. 6(a)-6(f) illustrate the results of artery vein separation algorithm on two different MRA data. FIGS. 6(a) and 6(d) show the arteries (labeled RED) and veins (labeled BLUE) depicted in volume rendering (VR) visualization; FIGS. 6(b) and 6(e) show a venous map; and, FIGS. 6(c) and 6(f) show an arterial map. (It should be understood that the items labeled RED and BLUE would typically be displayed in colors such as for example red and blue, respectively).

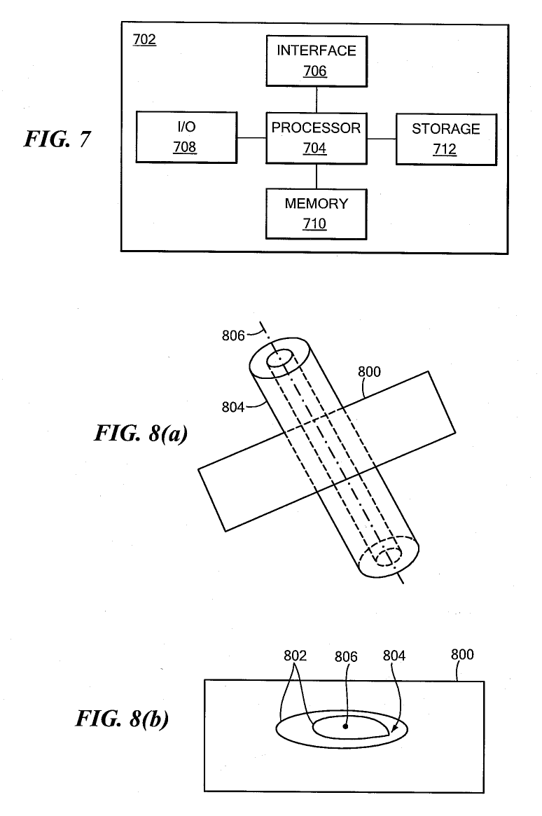

The method described above may be performed by an appropriately programmed computer. An appropriate computer may be implemented, for example, using well-known computer processors, memory units, storage devices, computer software, and other components. A high-level block diagram of such a computer is shown in FIG. 7. Computer 702 contains a processor 704 which controls the overall operation of computer 702 by executing computer program instructions which define such operation. The computer program instructions may be stored in a storage device 712 (e.g., magnetic disk) and loaded into memory 710 when execution of the computer program instructions is desired. Computer 702 also includes one or more interfaces 706 for communicating with other devices (e.g., locally or via a network). Computer 702 also includes input/output 708 which represents devices which allow for user interaction with the computer 702 (e.g., display, keyboard, mouse, speakers, buttons, etc.).

One skilled in the art will recognize that an implementation of an actual computer will contain other components as well, and that FIG. 7 is a high level representation of some of the components of such a computer for illustrative purposes. In addition, one skilled in the art will recognize that the processing steps described herein may also be implemented using dedicated hardware, the circuitry of which is configured specifically for implementing such processing steps. Alternatively, the processing steps may be implemented using various combinations of hardware and software. Also, the processing steps may take place in a computer or may be part of a larger machine (e.g., a medical imaging machine).

A number of embodiments of the invention have been described. Nevertheless, it will be understood that various modifications may be made without departing from the spirit and scope of the invention. Accordingly, other embodiments are within the scope of the following claims.

What is claimed is:

1. A method for artery segmentation from background in a patient, comprising:
performing modeling on a local vessel of the patient;
applying discrete centerline models to the modeled local vessel;
generating an ordered statistical front propagation on the discrete centerline models; and
generating arteries and veins from the generating ordered statistical front propagation generated on the discrete centerline models.

2. The method recited in claim 1 including separating, in the generated arteries and veins, the arteries from the veins using centerline models to the distance based watershed transforms.

3. A method for artery segmentation from background in a patient, comprising:
segmenting the vessels using local vessel modeling;
developing statistical front propagation and modeling from the local vessel modeling; and
using discrete centerline modeling.

4. The method recited in claim 3 wherein the segmenting of the vessels using local vessel modeling comprises: placing seed points on an image of a portion of the patient.

5. The method recited in claim 4 wherein the developing statistical front propagation and modeling from the local vessel modeling for the each seed point comprises: locally estimating vessel and surrounding background statistics by computing vessel orthogonal planes and corresponding cross-sectional boundaries; and segmenting vessels in a limited area partially based on a front propagation algorithm using the estimated statistics.

6. The method recited in claim 5 wherein the discrete centerline modeling comprises re-estimating vessel statistics using discrete fronts and surface filling.

7. The method recited in claim 6 wherein the method determines a measure of accuracy of each front using a discrete centerline model obtained by minimal path detection operating a distance map and re-starts partial segmentation from the front having the highest confidence measure representing the correct vessel.

8. The method recited in claim 7 including iteratively performing partial segmentation to segment arterial and venous vessels independently; each one of the arterial and venous vessels having a separate arterial and venous vessels map.

9. The method recited in claim 8 including combining the independently segmented arterial and venous vessels maps into a single map.

10. The method recited in claim 9 wherein the arterial and venous vessels in the single map are separated by a distance-based watershed transform using discrete centerline models between seeds used as the markers for the watershed transforms.

11. A method for artery segmentation from background in a patient comprising:
segmenting the vessels using local vessel modeling; and
developing statistical front propagation and modeling from the local vessel modeling.

12. The method recited in claim 11 wherein the segmenting of the vessels using local vessel modeling comprises: placing seed points on an image of a portion of the patient.

13. The method recited in claim 12 wherein the developing statistical front propagation and modeling from the local vessel modeling for the each seed point comprises: locally estimating vessel and surrounding background statistics by computing vessel orthogonal planes and corresponding cross-sectional boundaries; and segmenting vessels in a limited area partially based on a front propagation algorithm using the estimated statistics.

14. The method recited in claim 13 including using ordered front propagation, such propagation comprising applying discrete centerline modeling using the developed statistical front propagation and modeling comprising re-estimating vessel statistics using discrete fronts and surface filling followed by determining a measure of accuracy of each front using a discrete centerline model obtained by minimal path detection operating a distance map and re-starting partial segmentation from the front having the highest confidence measure representing the correct vessel.

15. The method recited in claim 14 including iteratively performing the partial segmentation and the ordered front propagation to segment arterial and venous vessels, independently; each one of the arterial and venous vessels having a separate arterial and venous vessels map.

16. The method recited in claim 15 including combining the independently segmented arterial and venous vessels maps into a single map.

17. The method recited in claim 16 including separating the arterial and venous vessels in the singe map using a distance-based watershed transform using discrete centerline models between seeds used as the markers for the watershed transforms.

18. A method for artery segmentation from background in a patient comprising:
placing seed points on an image of a portion of the patient
developing statistical front propagation and modeling from the local vessel modeling for the each seed point comprising locally estimating vessel and surrounding background statistics comprising computing vessel orthogonal planes and corresponding cross-sectional boundaries and segmenting vessels in a limited area partially based on a front propagation algorithm using the estimated statistics;
applying discrete centerline modeling using the developed statistical front propagation and modeling comprising re-estimating vessel statistics using discrete fronts and surface filling; and
determining a measure of accuracy of each front using a discrete centerline model obtained by minimal path detection operating a distance map and re-starting partial segmentation from the front having the highest confidence measure representing the correct vessel.

19. The method recited in claim 18 including iteratively performing the partial segmentation process to segment arterial and venous vessels independently; each one of the arterial and venous vessels having a separate arterial and venous vessels map.

20. The method recited in claim 19 including combining the independently segmented arterial and venous vessels maps into a single map.

21. The method recited in claim 20 including separating the arterial and venous vessels in the singe map by a distance-based watershed transform using discrete centerline models between seeds used as the markers for the watershed transforms.
